(19) United States
(12) Patent Application Publication
(10) Pub. No.: US 2025/0128040 A1
(43) Pub. Date: Apr. 24, 2025

(54) CEREBROSPINAL FLUID ACCESS METHODS AND DEVICES

(71) Applicants: Kuo Chao, Los Angeles, CA (US); Wei Chao, Cupertino, CA (US)

(72) Inventors: Kuo Chao, Los Angeles, CA (US); Wei Chao, Cupertino, CA (US)

(21) Appl. No.: 18/924,869

(22) Filed: Oct. 23, 2024

Related U.S. Application Data
(60) Provisional application No. 63/580,408, filed on Oct. 23, 2023.

(51) Int. Cl. A61M 39/02; A61M 25/00; A61M 25/09

(52) U.S. Cl. CPC A61M 39/0208; A61M 25/09; A61M 2025/0007; A61M 2202/0464

(57) ABSTRACT

A method of implanting a cerebrospinal fluid-access device is provided. The method can include interlocking the device onto a delivery assembly to keep the device fixed longitudinally on the delivery assembly, advancing the delivery assembly into the cerebrospinal fluid space, and releasing the interlock to allow the delivery assembly to be removed while leaving the device implanted in the cerebrospinal fluid.

### CROSS-REFERENCE TO RELATED APPLICATION

This application claims priority to U.S. Provisional Application No. 63/580,408, filed Oct. 23, 2023, and entitled “CSF INTERVENTIONS,” the disclosure of which application is hereby incorporated by reference in its entirety. The present application further incorporates hereby by reference in its entirety the disclosure of U.S. patent application Ser. No. 18/922,344, entitled “CEREBROSPINAL FLUID ACCESS DEVICES AND METHODS,” filed Oct. 21, 2024.

### BACKGROUND

Cerebrospinal fluid (CSF) is distributed around, and provides cushioning to, the brain and spinal cord. Access to a patient's CSF can be useful for diagnosing and treating a variety of physiological maladies. For example, infections such as spinal meningitis can be detected by sampling the CSF of a patient. The CSF space is also a convenient access point for introducing therapies that need to cross the blood-brain barrier, such as, for example chemotherapy agents.

Current methods of accessing the CSF are decades old and suffer from numerous deficiencies. The devices and methods disclosed herein are directed to novel ways of accessing the CSF of a person. The benefits of the devices and methods disclosed herein include increased treatment efficacy and patient comfort and safety compared to existing methods of accessing the CSF.

### SUMMARY

Disclosed herein are embodiments of a CSF-access device and methods of using the CSF-access device to perform diagnostic or therapeutic procedures on a patient. Also disclosed are methods of implanting a CSF-access device in a patient. In some arrangements, the CSF-access device can be implanted using an over-the-wire delivery assembly that passes through a central bore of the device. In some arrangements, the delivery assembly can comprise a coaxial arrangement of a guidewire within a delivery needle. In some arrangements, the delivery assembly can include a piercing stylet disposed within the delivery needle and configured to pierce a dura of the patient to gain access to a CSF space of the patient. In some aspects, methods of implanting a CSF-access device into the CSF space without using a guidewire are also disclosed herein.

Also disclosed are methods of accessing or removing a CSF-access device from a patient. In some arrangements, the proximal end of the device can include a magnetic collar configured to guide and otherwise facilitate coupling of the proximal end of the device to a distal end of a device-retrieval assembly. In some aspects, the CSF-access device and the device-retrieval assembly can be configured to form a reversible interlock with one another. The interlock can be configured such that in an engaged configuration of the interlock the CSF-access device and the device-retrieval assembly can be fixed longitudinally relative to one another, while in a disengaged configuration of the interlock the CSF-access device and the implant-retrieval assembly can move longitudinally relative to one another. In some arrangements, the proximal end of the CSF-access device can include a thread or groove that couples to a corresponding groove or thread of a device-retrieval assembly to allow the device-retrieval assembly to secure a coupling between the device and the device-retrieval assembly for removal of the device from the patient. In some arrangements, the proximal end of the CSF-access device can be a hook or ring structure that couples to a corresponding hook or ring or grasping structure of a device-retrieval assembly to allow the device-retrieval assembly to secure a coupling between the device and the device-retrieval assembly for removal of the CSF-access device from the patient.

Also disclosed are methods of closing an access site after removing a CSF-access device from the access site. In some aspects, the methods of closing an access site can be applied to close other CSF leak sites as well. In some arrangements, a device-closure assembly can include a balloon port configured to inflate a balloon and a sealant port configured to deliver a sealant to a distal end of the closure device. In some aspects, the device-closure assembly can be configured to inflate the balloon and seal the dura to prevent a sealant introduced through the sealant port from gaining access to the CSF-space. Upon the sealant curing, the balloon can be deflated and removed from the CSF-space in a low-profile configuration. In some arrangements, additional tissue sealant can be applied after deflation of the balloon and during withdrawal of the deflated balloon away from the CSF space.

### BRIEF DESCRIPTION OF THE DRAWINGS

Embodiments of the present disclosure will now be described hereinafter, by way of example only, with reference to the accompanying drawings in which:

FIG. 1 illustrates a prior art method of accessing the CSF of a patient.

FIG. 2 illustrates a near view of a prior art method of accessing the CSF of a patient.

FIG. 3A illustrates a needle access performed during a method of implanting a CSF-access device, according to some aspects of the present disclosure.

FIG. 3B illustrates a distal extension of a guidewire performed during a method of implanting a CSF-access device, according to some aspects of the present disclosure.

FIG. 3C illustrates an implant delivery performed during a method of implanting a CSF-access device, according to some aspects of the present disclosure.

FIG. 3D illustrates a CSF-access device implanted by a method of implanting a CSF-access device, according to some aspects of the present disclosure.

FIG. 4 illustrates a cross-sectional view of a CSF-access device mounted on a delivery assembly for implanting the CSF-access device, according to some aspects of the present disclosure.

FIG. 5A illustrates a method of implanting a CSF-access device, according to some aspects of the present disclosure.

FIG. 5B illustrates the CSF-access device of FIG. 5A implanted in a patient following removal of the delivery assembly, according to some aspects of the present disclosure.

FIG. 6A illustrates a method of using a transdermally-implanted CSF-access device to access the CSF space for an intrathecal diagnostic or therapeutic procedure, according to some aspects of the present disclosure.

FIG. 6B illustrates a method of accessing with a distal end of a instrumentation catheter a proximal hub of a subcutaneously-implanted CSF-access device, according to some aspects of the present disclosure.

FIG. 6C illustrates a distal-to-proximal end view of a distal end of the instrumentation catheter of FIG. 6B, according to some aspects of the present disclosure.

FIG. 6D illustrates a proximal-to-distal end view of a proximal end of the CSF-access device of FIG. 6B, according to some aspects of the present disclosure.

FIG. 7A illustrates an instrumentation catheter deployed through a transdermally-implanted CSF-access device, the instrumentation catheter being configured to implant intrathecally an electrode on a patient's brain and spinal cord, according to some aspects of the present disclosure.

FIG. 7B illustrates a subcutaneously-implanted CSF-access device configured as an interface hub for a system of electrodes implanted on a patient's brain and spinal cord, according to some aspects of the present disclosure.

FIG. 8A illustrates an instrumentation catheter deployed through a CSF-access device, the instrumentation catheter being configured to implant a miniature robot from the CSF space of a patient's brain and spinal cord, according to some aspects of the present disclosure.

FIG. 8B illustrates a CSF-access device configured to implant a miniature robot from the CSF space of a patient's brain and spinal cord, according to some aspects of the present disclosure.

FIG. 9A illustrates a CSF-access device configured to assist with placement of a retriever device into the CSF space, according to some aspects of the present disclosure.

FIG. 9B illustrates a device-retrieval catheter deployed through a CSF-access device, the device-retrieval catheter being configured to retrieve a miniature robot from the CSF space of a patient's brain and spinal cord, according to some aspects of the present disclosure.

FIG. 10A illustrates methods of treating a CSF leak including a CSF-venous fistula through a CSF-access device, according to some aspects of the present disclosure.

FIG. 10B illustrates methods of performing an imaging or biopsy procedure through a CSF-access device, according to some aspects of the present disclosure.

FIG. 10C illustrates methods of restoring or managing cerebral blood flow through a CSF-access device, according to some aspects of the present disclosure.

FIG. 10D illustrates further methods of performing an imaging or biopsy procedure through a CSF-access device, according to some aspects of the present disclosure.

FIG. 11 illustrates an implant-removal device configured to remove an implanted CSF-access device from a patient, according to some aspects of the present disclosure.

FIG. 12A illustrates an interlock mechanism for a CSF-access device and a implant-removal device, according to some aspects of the present disclosure.

FIG. 12B illustrates an interlock mechanism for a CSF-access device and a implant-removal device, according to some aspects of the present disclosure.

FIG. 12C illustrates an interlock mechanism for a CSF-access device and a implant-removal device, according to some aspects of the present disclosure.

FIG. 12D illustrates an interlock mechanism of a CSF-access device, according to some aspects of the present disclosure.

FIG. 12E illustrates an interlock mechanism of a CSF-access device, according to some aspects of the present disclosure.

FIG. 13 illustrates a cross-sectional view of a closure device repairing a CSF access site following removal of an implanted CSF-access device, according to some aspects of the present disclosure.

FIG. 14A illustrates a method of implanting a CSF-access device, according to some aspects of the present disclosure.

FIG. 14B illustrates a method of implanting a CSF-access device, according to some aspects of the present disclosure.

FIG. 14C illustrates a method of removing from a patient an implanted CSF-access device, according to some aspects of the present disclosure.

FIG. 14D illustrates a method of closing a CSF leak site such as an implant site following removal of an implanted CSF-access device, according to some aspects of the present disclosure.

### DETAILED DESCRIPTION

### Overview

The present disclosure relates generally to devices and methods for accessing a cerebrospinal fluid (CSF) of a patient. Also disclosed herein are delivery devices and methods for implanting a CSF-access device into a patient. In some aspects, the CSF-access device can be used to access the CSF and CSF space of the patient. In some arrangements, the devices and methods disclosed herein can be used to facilitate performing a diagnostic or therapeutic procedure on the patient. In some arrangements, the devices and methods disclosed herein can treat CSF leaks (e.g., CSF-venous fistulas). In some variations, the methods and devices can be used for intrathecal delivery of therapeutic agents, such as, for example, anti-cancer drugs, stem cells, trophic factors, vectors, energy, miniature robots. Also disclosed herein, are devices and methods directed to the removal and retrieval of a CSF-access device that has been implanted into a patient. Also disclosed are devices for closing CSF leakage sites such as, for example, tissue access sites following removal of an implanted CSF-access device.

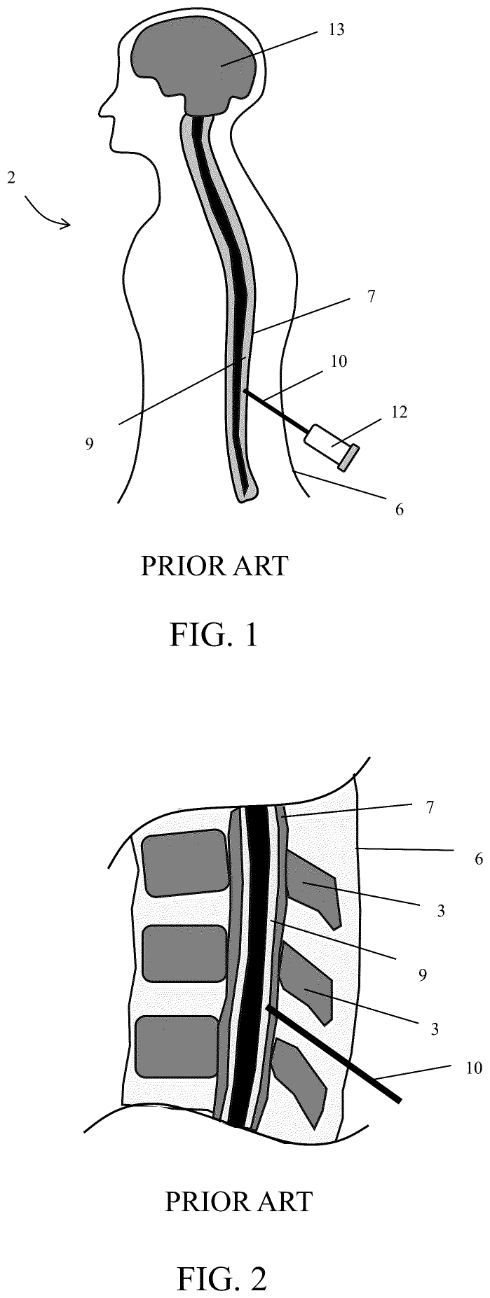

FIG. 1 illustrates a prior art method of accessing the CSF of a patient 2. The prior art method can include having the patient 2 lay on the patient's side or prone with the back of the patient 2 facing a medical practitioner. The medical practitioner can access the CSF of the patient 2 by inserting a needle 10 through the skin 6 of the patient 2, passing the needle 10 between two adjacent vertebrae 3, and penetrating the dura 7 to access the CSF space 9 of the patient 2. The proximal end 12 of the needle 10 (i.e., the end of the needle 10 that closest to the medical practitioner and most distant from the patient) can be configured to allow a CSF sample to be collected from the CSF space 9. FIG. 2 illustrates a prior art method for accessing a CSF space 9 with a needle 10 (e.g., a spinal needle, a needle with an inner stylet). The needle 10 can have a small profile and can be rigid such that the needle 10 can be passed through the skin 6 and between two adjacent vertebra 3 to pierce the dura 7 of the patient 2 and gain access to the CSF space 9.

### CSF-Access Device Implantation

Certain embodiments of the disclosure comprise implanting a CSF-access device into a patient to gain access to the CSF space of the patient, such methods being configured to integrate with a CSF-access device such as the CSF-access devices 100 disclosed herein or in U.S. patent application Ser. No. 18/922,344, entitled “CEREBROSPINAL FLUID ACCESS DEVICES AND METHODS,” filed Oct. 21, 2024, the entire contents of which are incorporated herein by reference.

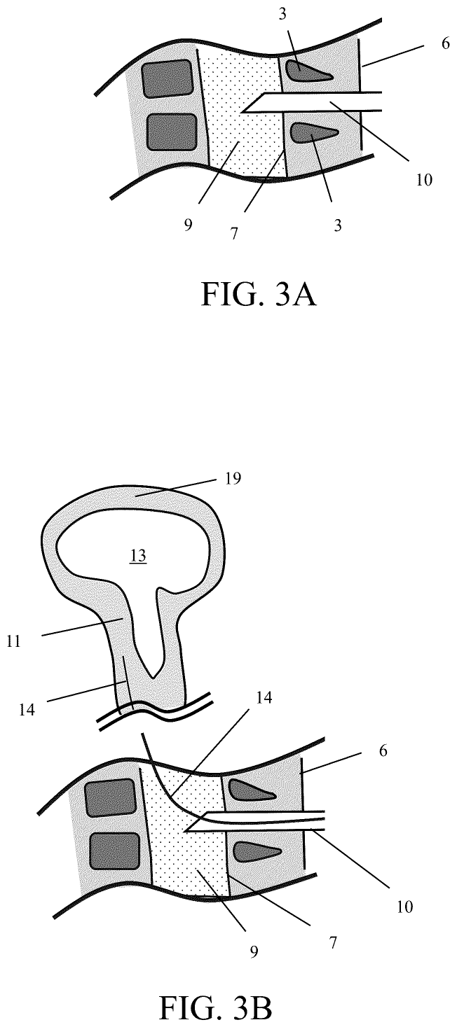

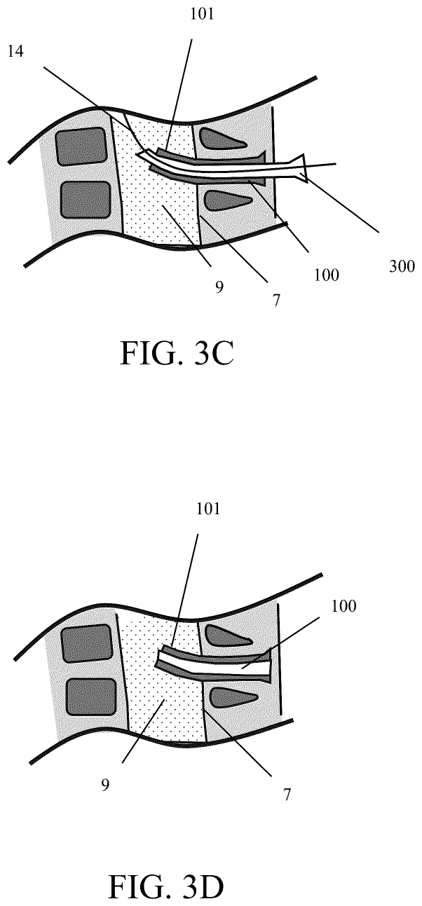

FIGS. 3A-3D depict an illustrative, non-limiting example of a method of implanting a CSF-access device 100, according to some aspects of the present disclosure. In some aspects, the method can use a modified Seldinger technique to implant the CSF-access device 100 over a guidewire. FIG. 3A shows that the implantation method can include using a needle 10 (e.g., spinal needle) with a small diameter and stiff proximal shaft to pierce the skin 6 and the dura 7 to gain access to the CSF space 9. In some arrangements, a stylet (not shown) can be advanced through a bore of the needle 10 to puncture tissue (e.g., skin 6, dura 7). FIG. 3B illustrates that following puncture of the dura 7 with the needle 10, a guidewire 14 can be inserted through the bore of the needle 10 and extended into the CSF space 9. The guidewire 14 can include a soft tip and can gradually grow stiffer in the proximal direction. In some arrangements, the guidewire 14 can be advanced into the subcranial space 11 that surrounds the brain 13, as depicted in the top portion of FIG. 3B. In some arrangements, the guidewire 14 can be extended into the cranial space 19. After the guidewire 14 has been placed in its desired location, the needle 10 can be withdrawn from the patient 2 while leaving the guidewire 14 in place. The placed guidewire 14 can then be available as a rail along which to pass a CSF-access device 100 or a delivery device configured to implant a CSF-access device 100 into the patient 2, as disclosed herein.

FIG. 3C illustrates a non-limiting, illustrative example of a CSF-access device 100 mounted onto a delivery assembly 300. In some aspects, the CSF-access device 100 and the delivery assembly 300 can be locked together and advanced along a previously-placed guidewire 14 to dispose the distal end 101 of the CSF-access device 100 in the CSF space 9. After the CSF-access device 100 is properly placed in its implanted position, the guidewire 14 and delivery assembly 300 can be removed from the bore of the CSF-access device 100 by withdrawing proximally the guidewire 14 and delivery assembly 300 through the proximal end 103 of the CSF-access device 100, as described herein. For example, the delivery assembly 300 can be guided to the CSF space 9 along a guidewire 14 left in place following the removal of the needle 10 (FIG. 3B) used to place the guidewire 14. FIG. 3D illustrates that after the CSF-access device 100 has been properly seated within the CSF space 9, the CSF-access device 100 can be decoupled from the delivery assembly 300 allowing the CSF-access device 100 to be left implanted in the patient 2 while the delivery assembly 300 and guidewire 14 can be removed from the patient 2. In some aspects, the CSF-access device 100 can be configured as a lumbar sheath that provides an access conduit for imaging, biopsy, and other therapeutic and diagnostic procedures, as described herein. In some arrangements, the CSF-access device 100 can be configured as a lumbar drain, a lumbar port, a lumbar shunt, or a lumbar electrode port, as described herein and in related U.S. patent application Ser. No. 18/922,344, entitled “CEREBROSPINAL FLUID ACCESS DEVICES AND METHODS,” filed Oct. 21, 2024.

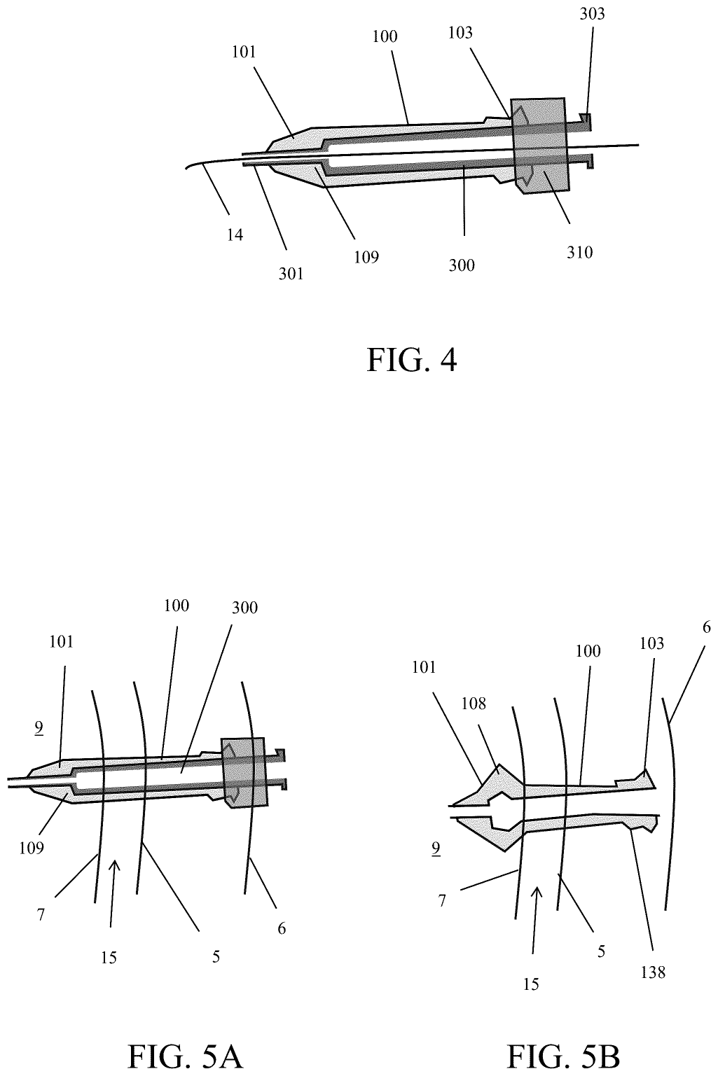

FIG. 4 depicts an illustrative, non-limiting example of a CSF-access device 100 mounted radially outward onto a delivery assembly 300 in preparation for implanting the CSF-access device 100 into a patient 2, according to some aspects of the present disclosure. In some aspects, the delivery assembly 300 can be configured as a hollow, needle-like structure that extends longitudinally through an inner bore of the CSF-access device 100. In the illustrated arrangement, the distal end 301 of the delivery assembly 300 extends distally beyond the distal end 101 of the CSF-access device 100. In some arrangements, the distal end 301 of the delivery assembly 300 can terminate within the CSF-access device 100 (e.g., proximal to the distal end 101 of the CSF-access device 100). The CSF-access device 100 can be configured to reversibly couple to the delivery assembly 300, allowing the CSF-access device 100 to move from a locked configuration to an unlocked configuration. In the locked configuration, the CSF-access device 100 and the delivery assembly 300 can be fixed (e.g., immovable) relative to one another, while in the unlocked configuration the CSF-access device 100 and the delivery assembly 300 can be moved relative to one another (e.g., separated from one another). FIG. 4 depicts an interlock 310 of the delivery assembly 300 that holds reversibly (e.g., through a luer-lock type of fitting) the proximal end 103 of the CSF-access device 100. The interlock 310 can be engaged (e.g., moved into a locked configuration) to secure the CSF-access device 100 longitudinally with respect to the delivery assembly 300 during implantation of the CSF-access device 100. Once the CSF-access device 100 is implanted, the interlock 310 can be disengaged (e.g., moved into an unlocked configuration) that allows the delivery assembly 300 to be removed (e.g., withdrawn proximally) from the CSF-access device 100.

The CSF-access device 100 can be mounted onto the delivery assembly 300 by advancing the CSF-access device 100 over the inner delivery assembly 300. The delivery assembly 300 can be used to implant a CSF-access device 100 configured as a shunt or as a port or as other configurations described herein, or in the related U.S. patent application Ser. No. 18/922,344, entitled “CEREBROSPINAL FLUID ACCESS DEVICES AND METHODS,” filed Oct. 21, 2024. In some arrangements, the delivery assembly 300 can have a distal end 301 that forms an interference fit with a bore constriction 109 at the distal end 101 of the CSF-access device 100. The proximal end 103 of the CSF-access device 100 can be held to a proximal end 303 of the delivery assembly 300 by a reversible interlock 310 (e.g., luer lock). The delivery assembly 300 can further include a hollow bore through which the guidewire 14 extends, as shown in FIG. 4. The guidewire 14 can be used to assist in guiding the delivery assembly 300 into proper placement for implantation. After the CSF-access device 100 has been properly placed and disengaged from the delivery assembly 300, the guidewire 14 and the delivery assembly 300 can be removed from the inner bore of the CSF-access device 100. In some arrangements, the delivery assembly 300 can be configured to implant a CSF-access device 100 without a guidewire 14. For example, the distal tip 301 can be configured to puncture and dilate intervening tissue to gain access to the CSF space 9.

FIGS. 5A and 5B depicts an illustrative, non-limiting example of a method of implanting a CSF-access device 100 using the delivery assembly 300 shown in FIG. 4, according to some aspects of the present disclosure. In some aspects, the CSF-access device 100 can be movable between a first form and a second form, with the first form being suited for delivery or implantation of the CSF-access device 100 and the second form being suited for using the use of the CSF-access device 100 for the intended purpose of implanting the CSF-access device. For the sake of simplicity, the first form will be referred to herein as a delivery configuration and the second form will be referred to as a deployed configuration. FIG. 5A shows the CSF-access device 100 in a delivery configuration and mounted onto a delivery assembly 300. FIG. 5B shows the CSF-access device in a deployed configuration in which the delivery assembly 300 has been detached and removed from the CSF-access device 100.

FIGS. 5A and 5B further illustrate the CSF-access device 100 can be configured to deploy a tissue anchor 108 upon release of the CSF-access device 100 from the delivery assembly 300. In some arrangements, the CSF-access device 100 can be held in tension by the interference fit at distal end 101 (e.g., a bore constriction 109) and the interlock 310 at proximal end 103. In some arrangements, the CSF-access device 100 can recoil longitudinally to deploy a tissue anchor 108. For example, FIG. 5A shows the delivery assembly 300 can be used to guide the distal end 101 of the CSF-access device 100 past the ligamentum flavum 5, through the epidural space 15, across the dura 7 and into the CSF space 9. The interlock 310 can then be moved to an unlocked configuration that releases the proximal end 103 of the CSF-access device 100 from connection to the delivery assembly 300. Removal of the delivery assembly 300 from the inner bore of the released CSF-access device 100 can allow the distal end 103 to recover elastically from the tension previously placed on the device 100 by the delivery assembly 300. FIG. 5B illustrates that the distal end 101 of the released CSF-access device 100 can recoil longitudinally toward the proximal end 103. The proximal end 103 can include anti-migration features such as ridges 138 to hold the proximal end 103 in place. The recoil of the distal end 101 toward the immobilized proximal end 103 can cause a tissue anchor 108 to deploy (e.g., by buckling) thereby holding the distal end 101 in the CSF space 9.

### CSF-Access Device Procedures

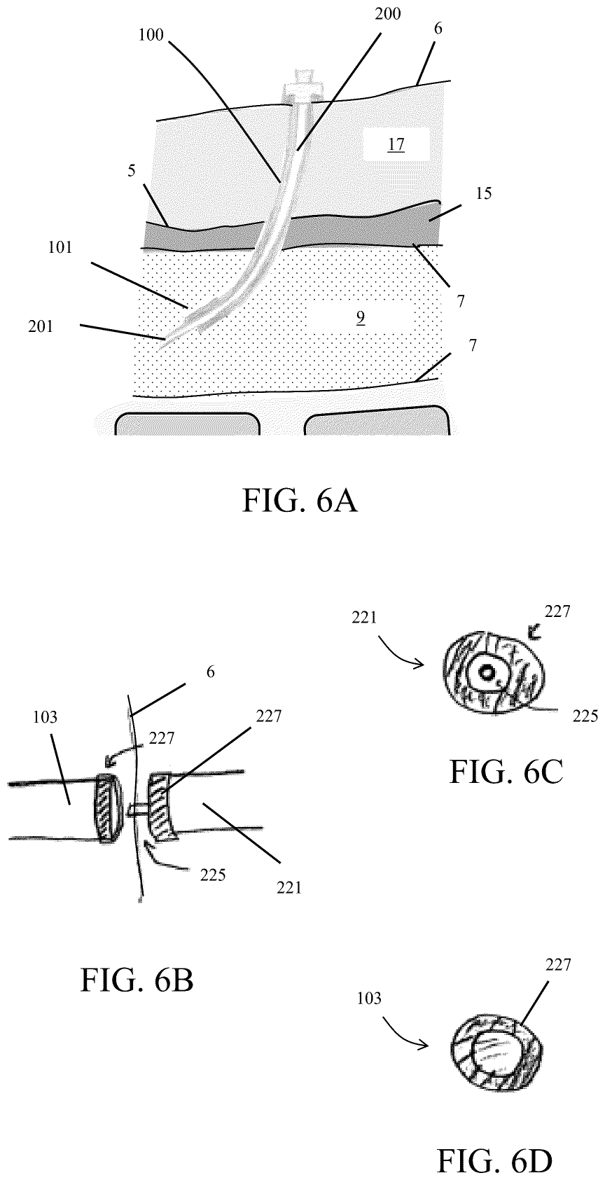

FIG. 6A illustrates a method of positioning a transdermally-implanted CSF-access device 100 configured for use to perform a diagnostic or therapeutic procedure on the patient 2, according to some aspects of the present disclosure. As described in related U.S. patent application Ser. No. 18/922,344, a dilator 200 can be used to position the distal end 101 of a CSF-access device 100. The distal end 201 of the dilator 200 can extend distally beyond the distal end 101 of the CSF-access device 100, as shown in FIG. 6A. In some arrangements, the dilator 200 does not extend completely longitudinally through the CSF-access device 100 but rather has the distal end 201 disposed proximal to the distal end 101 of the CSF-access device 100. The dilator 200 can have sufficient stiffness to apply torque to the distal end 101 of the CSF-access device 100 to rotate or otherwise pivot the distal end 101 into a desired orientation within the CSF space 9. The dilator 200 can be removed from the inner bore of the CSF-access device 100 to make the inner bore available for other medical instrumentation (e.g., an instrumentation catheter 220, an implant-retrieval device 500). In some aspects, the CSF-access device 100 can be configured as a sheath for an intrathecal diagnostic or therapeutic procedure, as shown in FIG. 6A. In some arrangements, the implanted CSF-access device 100 can be used as a conduit for introducing an endoscope into the CSF space 9. Alternative imaging modalities, such as, for example optical coherence tomography (OCT) and ultrasound, can use the conduit of the implanted CSF-access device 100 to access the CSF space 9. In some arrangements, the implanted CSF-access device 100 can be used to perform a biopsy such as, for example, a fine needle aspiration biopsy, a punch biopsy, a core biopsy. As described herein, the implanted CSF-access device 100 can be configured to facilitate surgical interventions to treat tumors, CSF abnormalities (e.g., arachnoid cyst, hydrocephalus, adhesions), or vascular malformations.

FIG. 6B illustrates a method of accessing a subcutaneously-implanted CSF-access device 100 with a medical-instrumentation catheter (e.g., an instrumentation catheter 220, an implant-retrieval device 500), according to some aspects of the present disclosure. FIG. 6B illustrates that the distal end 221 of the instrumentation catheter 220 can include a piercing feature 225 configured to pierce the skin 6 of the patient to gain access to the proximal end 103 of the subcutaneously-implanted CSF-access device 100. In some arrangements, the distal end 221 and the proximal end 103 can include magnetic features, such as for example, the magnetic collars 227 indicated in FIGS. 6B-6D. The magnetic collars 227 can be configured to magnetically interact to assist in aligning or securing the distal end 221 and proximal end 101 coaxially with one another.

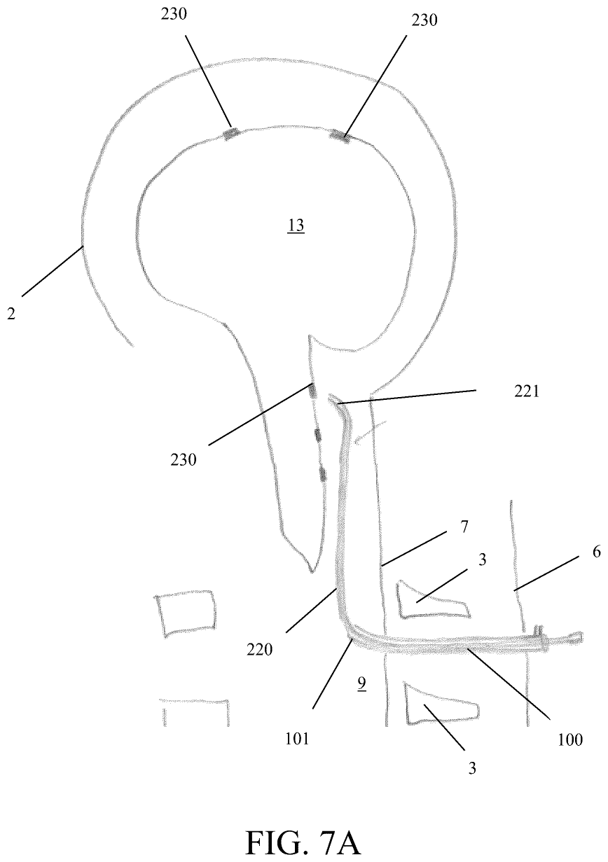

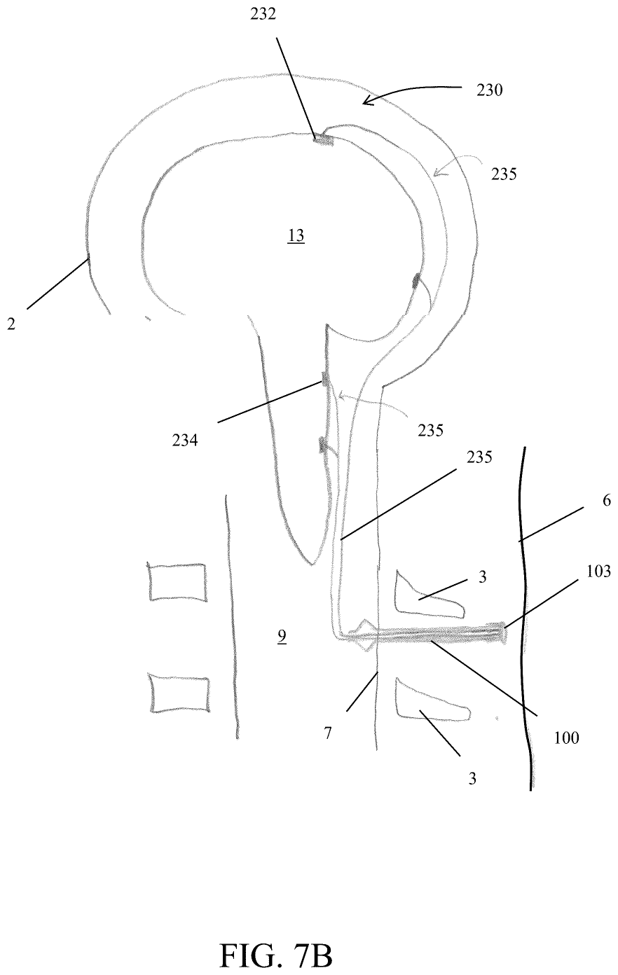

FIGS. 7A-7C depict non-limiting, illustrative examples of an implanted CSF-access device 100 being used to administer therapeutic or diagnostic procedures to a patient 2. CSF-access treatments can include: directed delivery treatments using catheters or miniature medical robots; cancer treatments such as delivery of drugs, vectors, energy; stroke treatments such as delivery of stem cells or trophic factors; degenerative disease treatments such as delivery of drugs, stem cells, or trophic factors to treat Alzheimer's or Parkinson's disease in a patient; treatment of vascular maladies such as miniature robots or surgical methods to treat aneurysm, arteriovenous malformation, treatment of CSF leaks (e.g., with miniature robots).

FIG. 7A illustrates the implanted CSF-access device 100 can be configured for multiple purposes such as a shunt and a transdermal conduit through which to access the CSF space 9. In FIG. 7A, an instrumentation catheter 220 has been extended through the bore of the CSF-access device 100 and into the subcranial space of the patient 2. The distal end 221 of the instrumentation catheter 220 can be configured to implant an electrode 230 on the brain 13 or spinal cord of the patient 2. In some arrangements, the instrumentation catheter 220 can be extended to bring the distal end 221 of the instrumentation catheter 220 into the cranial space, for example to implant an electrode 230 at the superior aspect of the brain 13 in FIG. 7A. In some aspects, the instrumentation catheter 220 can be guided by wires or can be steerable through torque. In some aspects, the implanted electrodes 230 can be configured to replace neural connections lost across lesions.

FIG. 7B illustrates the CSF-access device 100 can be configured as a subdermal port for electrodes 230 implanted on the brain 13 and/or spinal cord. In some aspects, the electrodes 230 can be configured as an input electrode 232 that transmits a signal to the brain 13 and/or spinal cord, the signal passing from the CSF-access device 100 to the brain 13 and/or spinal cord. In some aspects, the electrodes 230 can be configured as an output electrode 234 that transmits a signal from the brain 13 and/or spinal cord, the signal passing from the brain 13 and/or spinal cord to the CSF-access device 100. The electrode wiring 235 can be inserted through the CSF-access device 100 at the time of implantation. Access to the electrodes 230 and electrode wiring 235 through the CSF-access device can allow other electrodes to be inserted if an electrode malfunctions. The CSF-access device 100 and the electrodes 230 can enable a brain-computer interface, and/or a spinal cord-computer interface, to be established through the proximal end 103 of the CSF-access device 100, as illustrated in FIG. 7B. In some aspects, the electrodes 230 can be configured to provide a brain-to-spinal-cord bridge of electrical communication that crosses a defect or lesion and restores electrical communication across the defect or lesion.

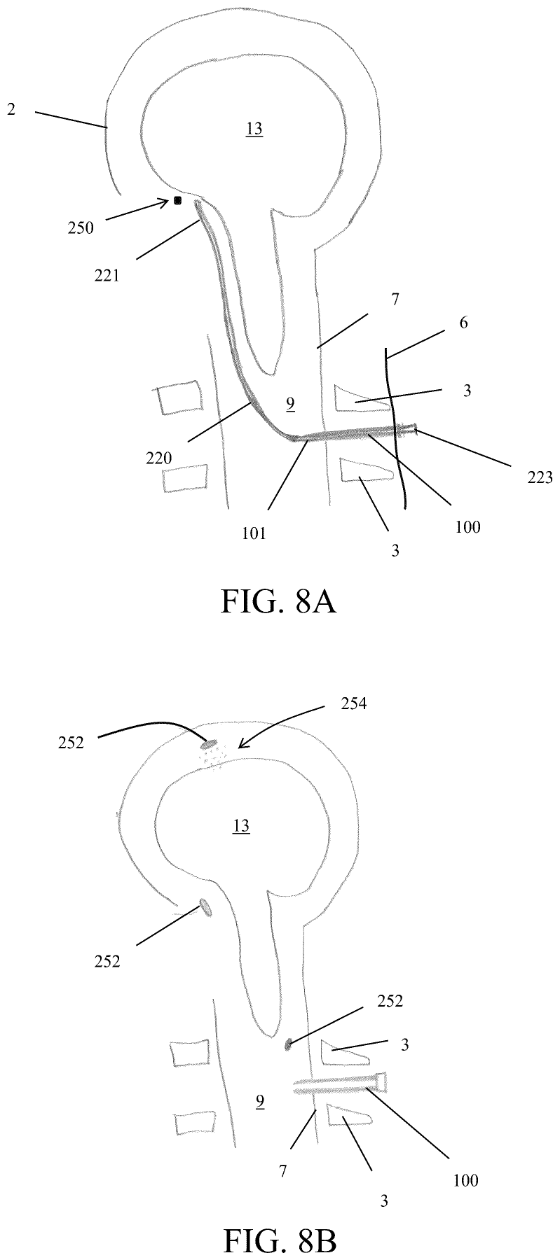

FIG. 8A illustrates the use of an implanted CSF-access device 100 for use in delivering a miniature robot 250 into the CSF space 9. In some aspects, the miniature robot 250 can be propelled or directed by a magnetic field. In some arrangements, the miniature robot 250 can be a configured to carry and release components such as pharmaceutical components. The miniature robots 250 can be configured to access anywhere the CSF space 9 allows the miniature robots 250 to gain access (e.g., the brain, the spinal cord) as well as gain access to surrounding structures like blood vessels and the dura 7. FIG. 8A shows the CSF-access device 100 can be configured as a transdermal port or shunt that provides a conduit for delivering the distal tip 221 of an instrumentation catheter 220 configured to navigate into the cranial or subcranial space while maintaining the proximal end 223 of the instrumentation catheter 220 accessible outside of the patient's skin 6. The distal end 221 can be directed by wire, by a magnetic field, or can include a torqueable tip. The instrumentation catheter 200 can be directed within the CSF space 9 and can further be configured to be embedded into tissue. The proximal end 223 of the instrumentation catheter 200 can be used to administer miniature robots 250 or other therapeutics through the CSF-access device 100 to the distal tip 221 of the catheter 220.

FIG. 8B depicts a larger carrier robot 252 can be used to transport a smaller released robot 254 (e.g., a nanobot), which the larger carrier robot 252 can release in an area of disease. In some arrangements, the miniature robot 250 (e.g., the carrier robot 252, the released robot 254) can be steered, propelled, or otherwise controlled by a magnetic field or a telecommunication modality such as, for example: radiofrequency, zigbee, wifi, cellular, Bluetooth, and near-field communication. The miniature robots 250 can be visualized using imaging modalities such as x-rays or MRI. The released robot 254, and other miniature robots 250, can be configured to deliver such items as: drugs, viral vectors, stem cells, and tissue factors. The CSF-access device 100 can be configured as a subdermal port that is used to access the CSF space 9 to release the carrier robot 252 or retrieve the released robots 254.

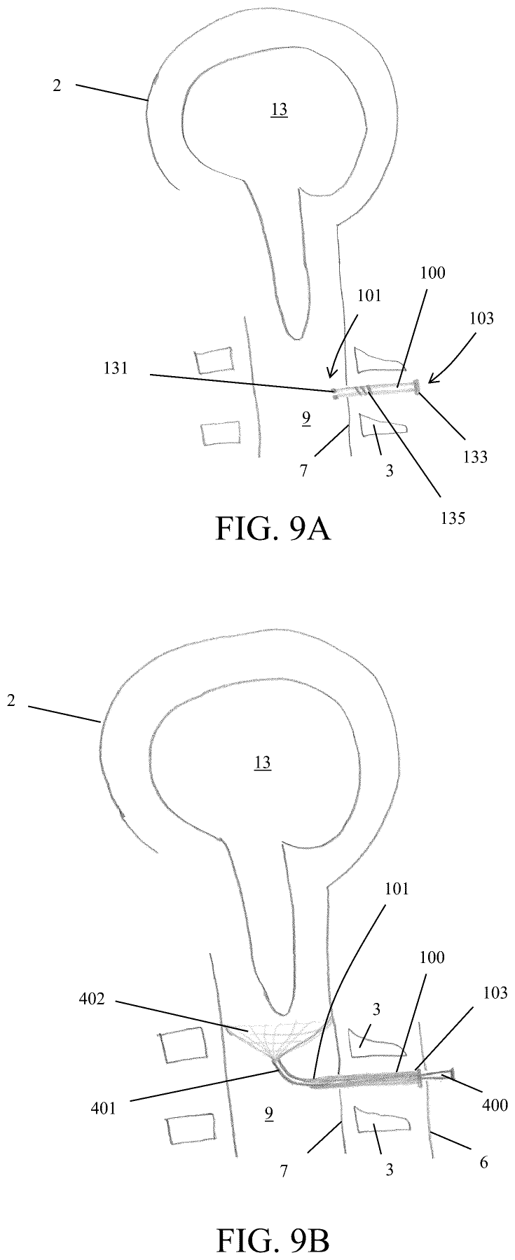

FIGS. 9A and 9B illustrate a CSF-access device 100 configured to assist with retrieval of a miniature robot 250 from the CSF space 9. FIG. 9A illustrates the CSF-access device 100 can include, singularly or any combination thereof, a magnetic distal tip 131, a magnetic proximal hub 133, and a magnetic central shaft 135. In some aspects, the magnets can be located to help direct a catheter-based device through the lumen of the implanted CSF-access device 100. In some arrangements, using an electromagnetic current can help direct the catheter-based device through the implanted CSF-access device 100 and can allow to turn on and off and various power. In some aspects, the electromagnetic current can change the strength and direction of the magnetic field based on the current. In some arrangements, the magnetic distal tip 131 can be used to attract miniature robots 250 that were previously released into the CSF space 9. The magnetic central shaft 135 can be used to help couple the CSF-access device 100 to a retrieval device that is inserted through the central bore of the CSF-access device 100. The magnetic proximal hub 133 can be used to help guide a robot-retrieval device 400 to the central bore of the implanted CSF-access device 100 or to couple the retrieval device to the implanted CSF-access device 100.

FIG. 9B illustrates a robot retrieval device 400 can be deployed through a CSF-access device 100 and into the CSF space 9. The retrieval of the miniature robots 250 can be done using nets or filters further guided by MRI, x-ray, or CT. For example, FIG. 9B illustrates a mesh 402 can be deployed from a distal end 401 of the robot retrieval device 400. In some arrangements, the mesh 402 can be magnetic to attract the miniature robots 250 previously-released into the CSF space 9. In some arrangements, the mesh 402 can be replaced with another suitable filtering structure such as a net, a rod, a plurality of rods, a web, a sieve.

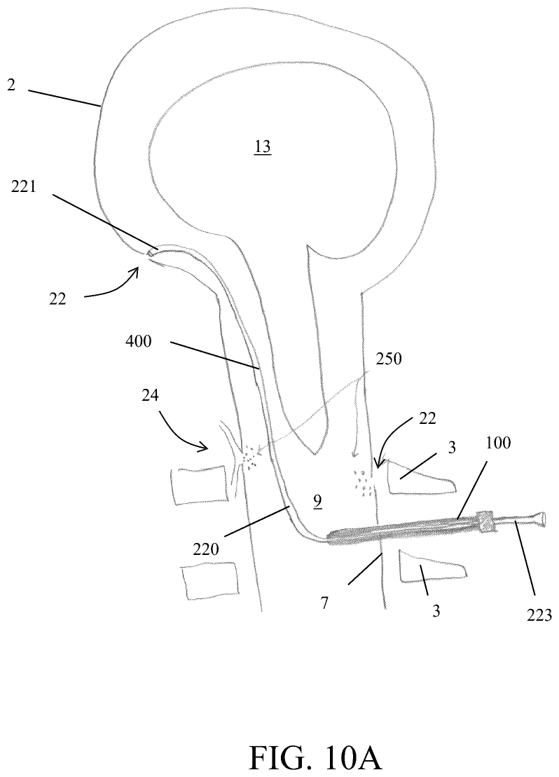

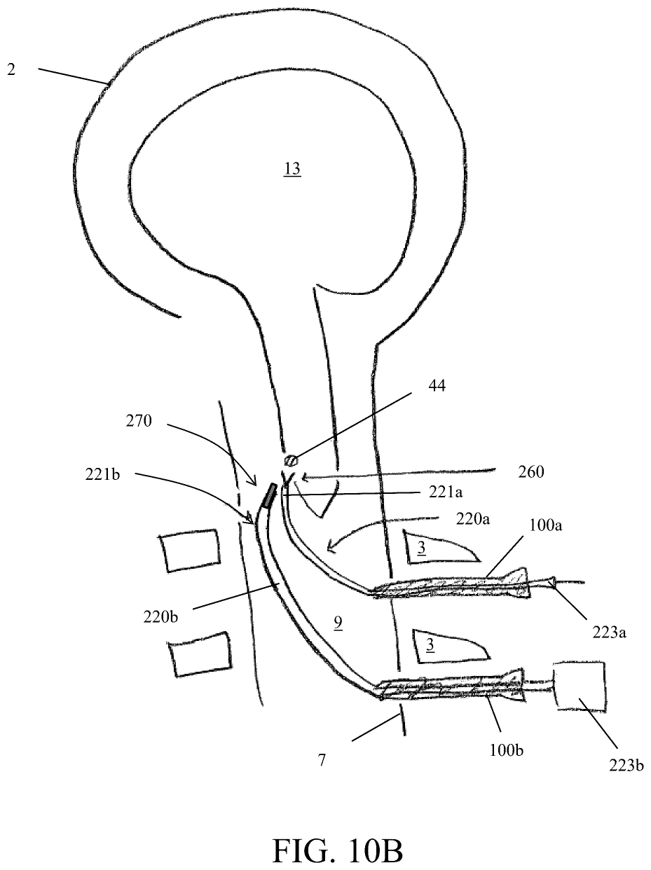

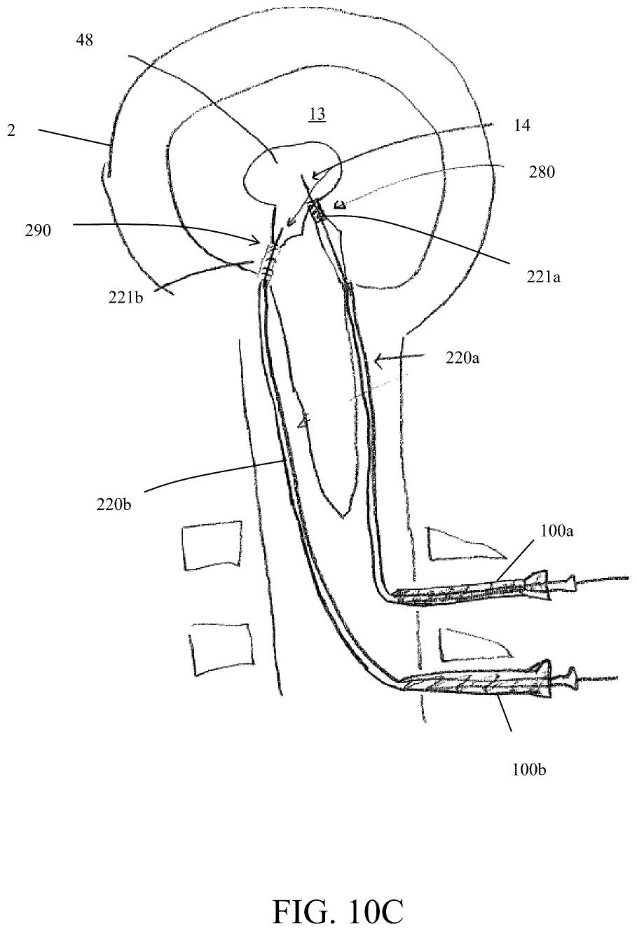

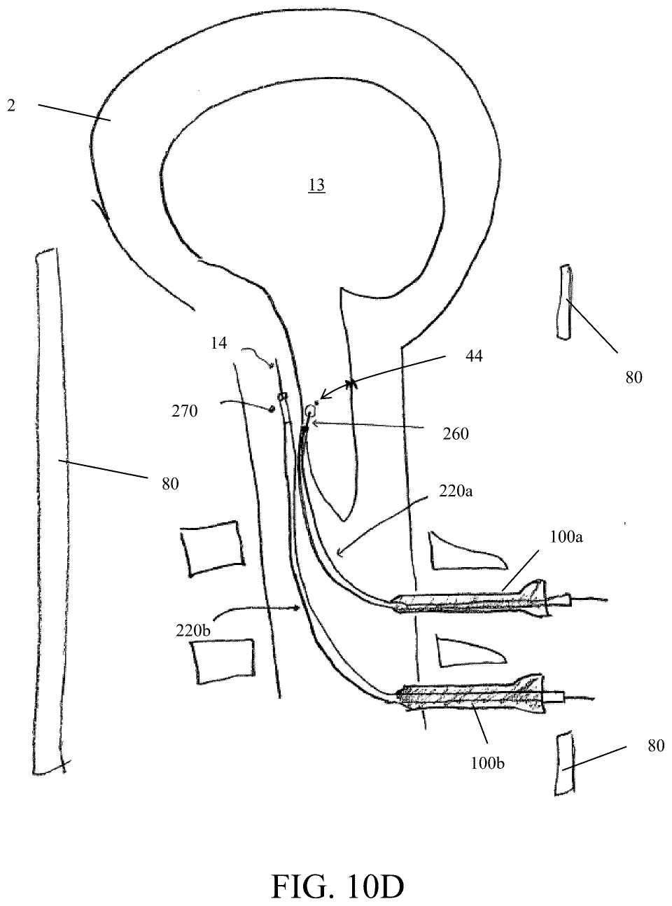

FIG. 10A illustrates a CSF-access device 100 being used to introduce an instrumentation catheter 220 configured to repair a leak site in the CSF space 9. FIG. 10A illustrates further the suitability of the CSF-access device 100 to assist with repair of a CSF venous fistula 24, as described herein. In FIG. 10A, the distal end 221 of the instrumentation catheter 200 has been guided (e.g., by wire, by magnetic field, by torque manipulation) to a leak site 22 of the CSF space 9. The distal end 221 can be used to introduce medical instruments to seal (e.g., suture close) the leak site. The CSF-access device 100 can enable intrathecal repair of a leak site 22 or of a CSF-venous fistula 24, which can help overcome the problem of CSF-venous fistulas 24 being difficult to find through approaching the fistula from the venous side of the vasculature.

FIG. 10A further illustrates that miniature robots 250 can be released from the instrumentation catheter 200 and can be directed (e.g., by magnetic field) to a leak site 22 or to a CSF-venous fistula 24. In some arrangements, the miniature robots 250 can be configured to congregate at a leak site 22 or a CSF-venous fistula 24. The miniature robots 250 can be further configured to be activated to release tissue-active components or substances that promote clotting. Although not shown in FIG. 10A, in some arrangements the distal end 221 of the instrumentation catheter 200 can be directed to a CSF-venous fistula to introduce medical instruments or miniature robots 250 to seal the CSF-venous fistula.

FIG. 10B illustrates a first CSF-access device 100a being used to introduce a first instrumentation catheter 220a configured to take a biopsy of a lesion 44. FIG. 10B further illustrates a second CSF-access device 100b being used to introduce a second instrumentation catheter 220b configured for imaging within the CSF space 9. FIG. 10B illustrates that multiple CSF-access devices 100 can be used together to perform a therapy on a patient 2. However, either instrumentation catheter 220a,b can be used without requiring the use of the other, as described herein. The distal end 221a of the first instrumentation catheter 220a can be configured to include (or to deliver) a tissue-collection feature 260 to the site of the lesion 44. In the illustrated embodiment, the tissue-collection feature 260 is shown as a scissoring device. In some arrangements, the tissue-collection feature 260 can be configured as a clamp, a scissors, a needle, a punch, or a shaving element. The distal end 221b of the second instrumentation catheter 220b can be configured to include (or to deliver) a tissue-imaging feature 270 to the site of the lesion 44. In the illustrated embodiment, the tissue-imaging feature 270 is depicted as an endoscope. In some arrangements, the tissue-imaging feature 270 can be configured as an ultrasound probe or other suitable imaging device.

FIG. 10C illustrates a first CSF-access device 100a being used to introduce a first instrumentation catheter 220a configured for performing a stenting procedure within the cerebral tissue. FIG. 10C further illustrates a second CSF-access device 100b being used to introduce a second instrumentation catheter 220b configured to establish an opening in the cerebral tissue (e.g., ventriculostomy). FIG. 10C illustrates the CSF-access devices 100a,b can be used together to perform a therapy on a patient 2. However, either instrumentation catheter 220a,b can be used without requiring the use of the other, as described herein. The distal end 221a of the first instrumentation catheter 220a can be configured to include (or to deliver) a stenting feature 280 to the brain 13. In the illustrated embodiment, the stenting feature 280 is shown as an expandable cylindrical wireframe stent. The distal end 221b of the second instrumentation catheter 220b can be configured to include (or to deliver) a channel-creation feature 290 configured to create an opening in the tissue of the brain 13, such as, for example to access the ventricles 48. In the illustrated embodiment, the channel-creation feature 290 is depicted as a tissue-coring device. In some arrangements, the channel-creation feature 290 can be configured as a cutting device or an expandable feature (e.g., an angioplasty device).

FIG. 10D is similar to FIG. 10B in that it illustrates a first CSF-access device 100a being used to introduce a first instrumentation catheter 220a configured to take a biopsy of a lesion 44 and a second CSF-access device 100b being used to introduce a second instrumentation catheter 220b configured for imaging within the CSF space 9. FIG. 10D further illustrates that a patient-external medical instrument 80 can be used to assist with procedures performed with the CSF-access device 100. For example, the patient-external medical instruments 80 can be an MRI, CT, or X-ray imaging device that assists with the placement of the instrumentation catheters 220a,b. FIG. 10D illustrates that multiple CSF-access devices 100 can be used together with patient-external medical instruments 80 to perform a therapy on a patient 2. However, either instrumentation catheter 220a,b can be used without requiring the use of the other, as described herein. For example, the first instrumentation catheter 220a could include an embedded marker or an insertable stylet that is detectable by the patient-external medical instrument 80. The patient-external medical instrument 80 can be used to guide the catheter 220a to the lesion 44, with both the lesion 44 and the catheter 220a being visible with the imaging modality applied by the patient-external medical device 80.

### CSF-Access Device Removal

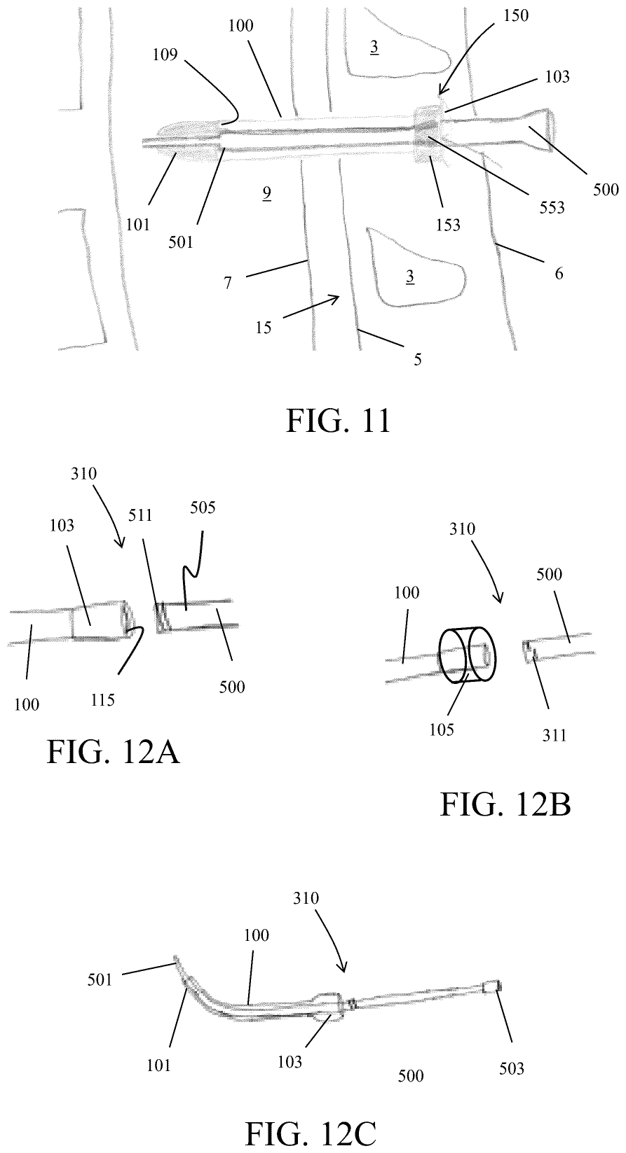

FIG. 11 depicts an illustrative, non-limiting example of a method of retrieving an implanted CSF-access device 100 from a patient 2 using an implant-retrieval device 500, according to some aspects of the present disclosure. In some arrangements, the implant-retrieval device 500 can be used to retrieve a venous access device 100 such as, for example, the venous access device shown in FIG. 5C of the related U.S. application Ser. No. 18/922,344. In certain variants, there can be a mark on the patient's skin (e.g., a tattoo) to indicate the approximate location of a proximal end 103 of the implanted CSF-access device 100. The proximal end 103 may contain a magnet 150 or echogenic marker to help locate the proximal end 103 of the implanted CSF-access device 100. As shown in FIG. 11, the implant-retrieval device 500 can be shaped generally as a tapered needle sized to fit within the central bore of the implanted CSF-access device 100. The proximal end 103 of the CSF-access device 100 can include a proximal magnetic element 153 (e.g., a magnetic collar) that, in some arrangements, helps guide the distal end 501 of the implant-retrieval device 500 into the central bore of the implanted CSF-access device 100. In some configurations, the proximal magnetic element 153 can be configured to form an interlocking mechanism with a corresponding magnetic feature 553 disposed on the implant-retrieval device 500 to assist in securing the implanted CSF-access device 100 to the implant-retrieval device 500 in anticipation of removal of the implanted CSF-access device 100. In some arrangements, the distal end 501 of the implant-retrieval device 500 can be sized to collide with a bore constriction 109 of the implanted CSF-access device 100, as indicated in FIG. 11, allowing the implant-retrieval device 500 to extend the implanted CSF-access device 100 longitudinally into a low-profile configuration (e.g., a delivery configuration), as described herein with regard to FIGS. 5A and 5B.

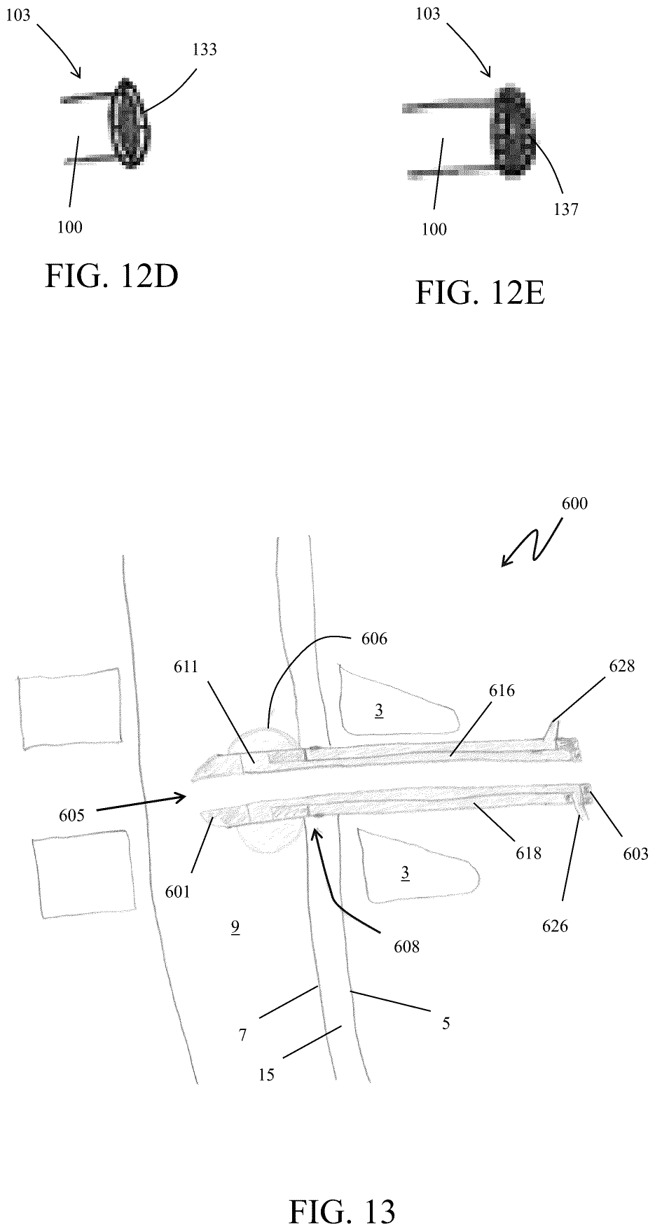

FIGS. 12A-12E depict non-limiting, illustrative examples of a configuration of an interlock 310 between a proximal end of an implanted CSF-access device and a distal end of an implant-retrieval device. FIG. 12A illustrates the interlock 310 can be formed by mating an external thread 511 disposed on an outer surface 505 of the implant-retriever device 500 with a corresponding internal thread 115 disposed on the proximal end of the inner bore of the CSF-access device 100. In some arrangements the retriever device 100 can include a magnetic thread or a magnetic feature adjacent to the thread that interacts magnetically with a corresponding magnetic thread or feature disposed on the CSF-access device 100 to help seat the implant-retriever device 500 properly within the CSF-access device 100 to form the interlock 310 therebetween. FIG. 12B illustrates that the interlock 310 can be formed by the interaction of a protrusion disposed on one of the CSF-access device 100 or the retriever device interacting with a corresponding groove 311 disposed on the other of the CSF-access device 100 or the retriever device. FIG. 12B further illustrates that the interlock 310 can include a scaling membrane 105 that covers the central bore at the proximal end 103 as described with regard to the CSF-access device 100 disclosed in U.S. patent application Ser. No. 18/922,344, entitled “CEREBROSPINAL FLUID ACCESS DEVICES AND METHODS,” filed Oct. 21, 2024. FIG. 12C illustrates that the interlock 310 need not be disposed on the proximal and distal ends, respectively, of the CSF-access device 100 and the implant-retrieval device 500. In FIG. 12C, the interlock 310 is shown approximately mid-length longitudinally on the implant-retriever device 500 that extends distally beyond the distal end 101 of the implanted CSF-access device 100. FIGS. 12D and 12E illustrate that the proximal end 103 of the CSF-access device 100 can include an bore-external feature that assists in forming the interlock 310 with an implant-retrieval device 500. In FIG. 12D, the proximal end 103 of the CSF-access device 100 includes an open frame structure 133 that can be used to form an interlock 310 with a hook-like feature (not shown) disposed on the implant-retrieval device 500. FIG. 12E illustrates the proximal end 103 can include a ring-like structure 137 that completely surrounds circumferentially the central bore of the CSF-access device 100. The ring-like structure 137 can be used to form the interlock 310 by providing an edge by which a gripping feature disposed on the implant-retrieval device 500 can grab and secure the ring-like structure. In some arrangements, the open-frame structure 133 or the ring-like structure 137 can be made from a magnetic material to assist in the formation of the interlock 310.

FIG. 13 depicts a non-limiting, illustrative example of a closure device 600, according to some aspects of the present disclosure. The closure device 600 can be used to repair a CSF-leak site such as, for example, tissue damage left behind after an implanted CSF-access device 100 has been removed from the dura 7. In some aspects, the closure device 600 can be configured to apply a tissue sealant to seal or repair puncture holes left by the implanted CSF-access device 100 in the ligamentum flavum 5 or the dura 7. In some arrangements, the closure device 600 can comprise a needle-like structure having concentrically arranged channels disposed in the wall of the needle-like structure, as described herein. In some arrangements, the closure device 600 can include an open central bore 605 that can be used to guide the closure device along a guidewire (not shown) that has been left in place after removal of the implanted CSF-access device 100. The closure device 600 can include a balloon 606 at a distal end 601 of the closure device 600. The interior space of the balloon 606 can be fluidically connected through an inflation orifice 611 to an inflation channel 616 that terminates at an inflation port 626 disposed on a proximal end 603 of the closure device 600. The closure device 600 can further include an outflow orifice 608 disposed on an outer surface of the closure device 600 and fluidically connected to an injection channel 618 that runs longitudinally along the closure device 600 and terminates at an injection port 628 disposed at the proximal end 603 of the closure device 600, as indicated in FIG. 13. The injection port 628 can be used to inject contrast or air to help properly place the closure device 600 in the epidural space 15. Once properly placed, tissue sealant (e.g., blood, gel, fibrin, other sealants) can be injected through the injection port 628, along the injection channel 618 to exit at the outflow orifice 608. Alternatively and additionally, tissue sealant can be injected through the central bore 605 as the closure device 600 is withdrawn across the dura 7 and into the epidural space 15. Tissue sealant can be injected through the central bore 605 again after the closure device has been further withdrawn proximally to cross the ligamentum flavum 5.

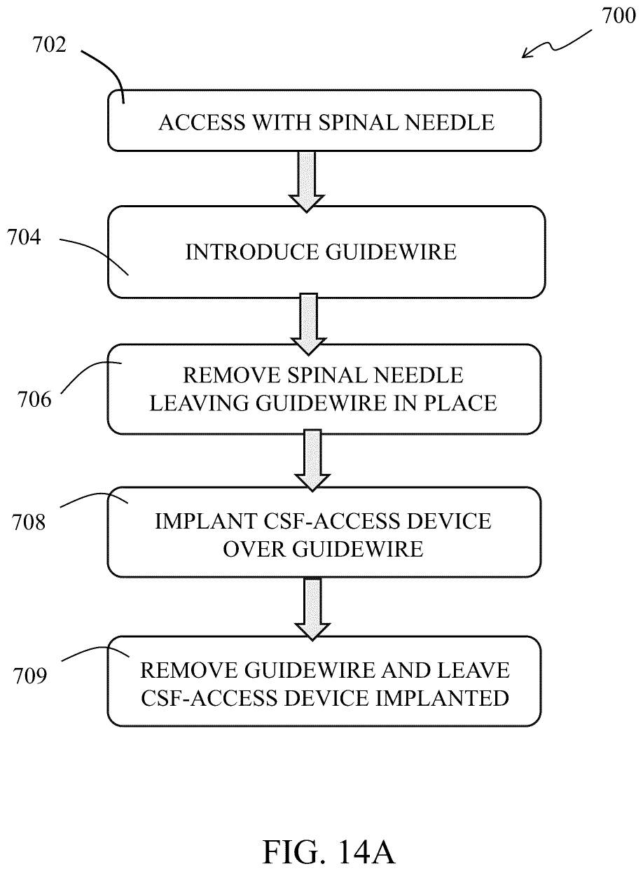

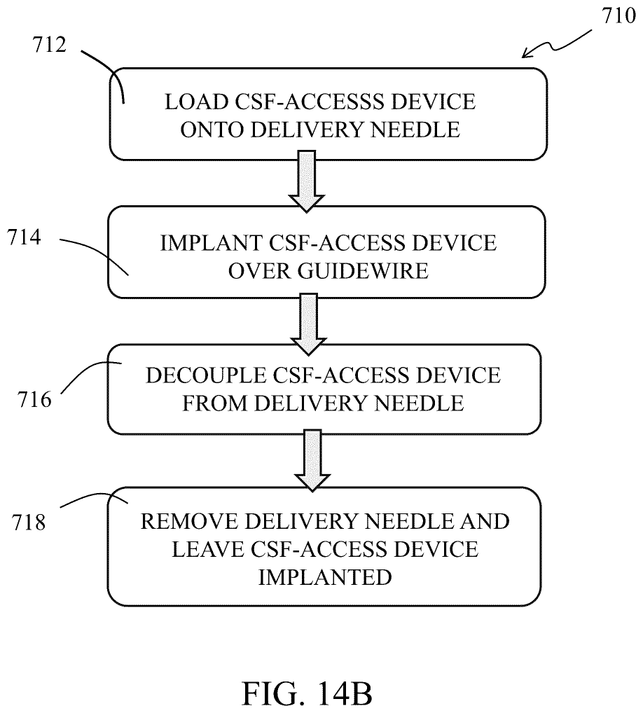

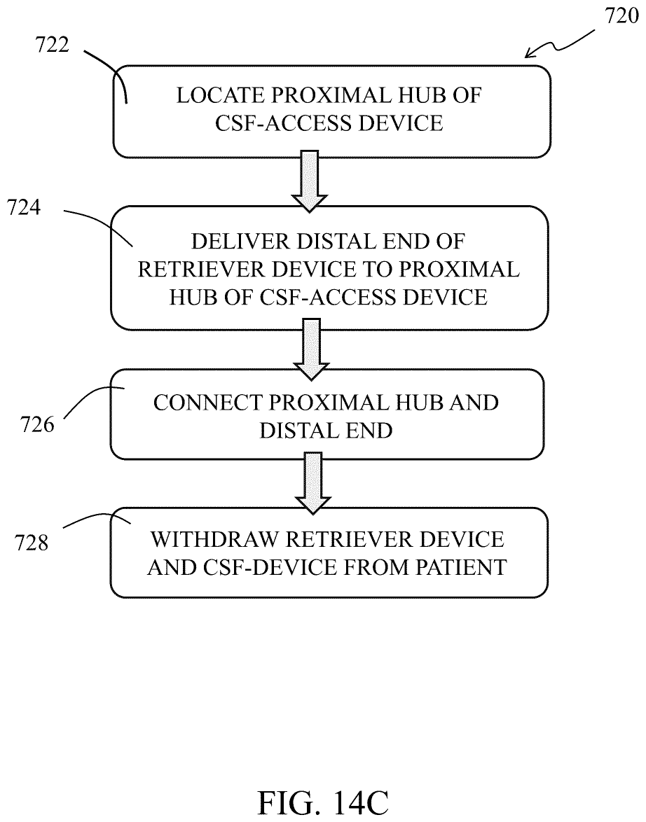

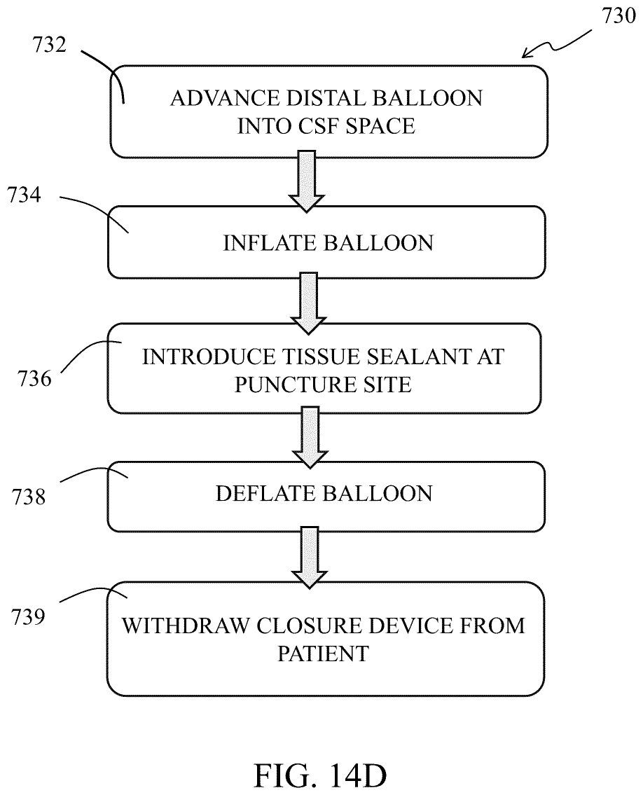

FIG. 14A illustrates a method 700 of implanting a CSF-access device 100, according to some aspects of the present disclosure. In some aspects, the method 700 can include a CSF-access step 702 in which a thin, rigid, hollow needle with inner stylet 10 (FIG. 3A) is advanced through the patient's skin 6, between adjacent vertebrae 3, through the ligamentum flavum 5, the epidural space 15, and the dura 7 to bring the distal end of the needle 10 into the CSF space 9. The method 700 can further include a guidewire-introducing step 704 in which the distal end of a guidewire is advanced distally thorough the central bore of the needle 10 to bring the distal end of the guidewire into the CSF space 9. The method 700 can further include a needle-removal step 706 in which the needle 10 is removed from the guidewire by moving the needle proximally along the guidewire to bring the distal end of the needle 10 longitudinally past the proximal end of the guidewire, leaving the distal end of the guidewire in place in the CSF space 9. The method 700 can further include an implant-placement step 708 in which the CSF-access device 100 is mounted on a delivery assembly, as described herein, and advanced along the guidewire 14 to bring the distal end 101 of the CSF-access device 100 into the CSF space 9. The method 700 can further include a guidewire-removal step 709 in which the guidewire 14 and the delivery assembly are removed from the patient by withdrawing proximally the guidewire 14 and the delivery assembly proximally out of the central bore of the CSF-access device 100, leaving the CSF-access device 100 implanted in the CSF space 9 of the patient 2. In some arrangements, the guidewire-removal step 709 can include disengaging an interlock 310 to allow the delivery assembly to move longitudinally with respect to the CSF-device 100, as described herein.

FIG. 14B illustrates a method 710 of implanting a CSF-access device 100, according to some aspects of the present disclosure. In some arrangements, the method 710 can include an implant-loading step 712 in which the CSF-access device 100 can be loaded onto and interlocked with a delivery device (e.g., delivery assembly 300). In some arrangements, the CSF-access device 100 can be configured to be held by the delivery device in a small-profile configuration (e.g., delivery configuration) in which an outer dimension of the CSF-access device 100 is reduced compared to a large-profile configuration (e.g., deployed configuration). The CSF-access device 100 can further be configured to move or be movable from the small-profile configuration to the large-profile configuration upon the CSF-access device 100 being de-coupled from the delivery device. The method 710 can further include an implant-assembly-advancement step 714 in which the delivery device is advanced along a guide wire coupled with a CSF-access device 100 that is held in the small-profile configuration. In some arrangements, the implant-assembly-advancement step 714 can be performed without the use of a guidewire. In other words, the delivery device can be configured, in some arrangements, to hold the CSF-access device 100 longitudinally fixed as the distal tip of the delivery device guides the CSF-access device 100 past the vertebrae 3, the ligamentum flavum 5, the dura 7, and into the CSF space 9. The delivery device can be advanced along the guidewire (or without the guidewire) distally to bring a distal end 101 of the CSF-access device 100 into the CSF space 9, as described herein. The method 710 can further include an implant-decoupling step 716 in which the CSF-access device 100 is released from the interlock with the delivery device, allowing the CSF-access device 100 to move from the small-profile configuration to the large-profile configuration. The method 710 can further include a delivery-device-removal step 718 in which the delivery device is withdrawn from the patient 2 proximally along, or along with, the guidewire, leaving the CSF-access device 100 implanted in the patient 2.

FIG. 14C illustrates a method 720 of removing an implanted CSF-access device 100, according to some aspects of the present disclosure. In some arrangements, the method 720 can include a hub-locating step 722 in which a proximal end 103 of the implanted CSF-access device 100 is located spatially. In some variants, a mark (e.g., tattoo) can be applied to the skin 6 of the patient 2 to help locate the proximal end 103. In some variants, the CSF-access device 100 can include a magnetic or echogenic element to assist in locating the proximal end 103, as described herein. In some aspects, the method 720 can include a device-implant-introducing step 724 in which the distal end 503 of an implant-retrieval device 500 is advanced distally into or past a proximal end 103 of the implanted CSF-access device 100. The method 720 can further include a coupling step 726 in which the implanted CSF-access device 100 is secured to the implant-retrieval device 500. In some aspects, a magnetic connection can be established to secure the implanted CSF-access device 100 to the implant-retrieval device 500. The method 720 can further include a removal step 728 in which the implant-retrieval device 500 is withdrawn proximally from the patient 2, bringing along the implanted CSF-access device 100 that is coupled to the implant-retrieval device 500.

FIG. 14D illustrates a method 730 of closing a tissue site following removal of an implanted CSF-access device 100, according to some aspects of the present disclosure. The method 730 can include a balloon-advancement step 732 in which a balloon 606 mounted on a closure device 600 can be advanced into the CSF space 9. In some arrangements, the closure device 600 can have a hollow bore that extends from a distal end 601 of the closure device 600 to a proximal end 603 of the closure device 600, allowing the closure device 600 to be advance over an existing guidewire. The method 730 can further include a balloon-inflation step 734 in which the balloon 606 can be inflated by passing an inflation fluid (e.g., saline) through an inflation channel of the closure device 600. In some arrangements, the method 730 can further include a tissue-sealant-releasing step 736 in which tissue sealant is passed through a lateral channel of the closure device and out an outer surface the closure device 600 and into the epidural space 15 of the patient. The method 730 can further include a balloon-deflation step 738 in which the balloon 606 is deflated to bring the balloon 606 in a low-profile configuration. In some aspects, the balloon 606 can be deflated after the tissue sealant has had sufficient time to solidify, cure, or otherwise stabilize. The method 730 can further include a device-removal step 739 in which the closure device 600 can be removed by withdrawing proximally the closure device 600 away from the CSF space 9 and out of the patient 2.

### Other Variations and Terminology

While certain embodiments have been described, these embodiments have been presented by way of example only and are not intended to limit the scope of protection. Indeed, the novel methods and systems described herein may be embodied in a variety of other forms. It will be understood by those skilled in the art that the present disclosure extends beyond the specifically disclosed embodiments to other alternative embodiments or uses and obvious modifications and equivalents thereof, including embodiments which do not provide all of the features and advantages set forth herein. Furthermore, various omissions, substitutions, and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed; others may be added. Accordingly, the scope of the present disclosure is not intended to be limited by the specific disclosures of preferred embodiments herein and may be defined by claims as presented herein or as presented in the future. The language of the claims is to be interpreted broadly based on the language employed in the claims and not limited to the examples described in the patent specification of during prosecution of the application, which examples are to be construed as non-exclusive.

Features, materials, characteristics, or groups described in conjunction with a particular aspect, embodiment, or example are to be understood to be applicable to any other aspect, embodiment, or example described herein unless incompatible therewith. All of the features disclosed in this specification (including any accompanying claims, abstract, and drawings), or all of the steps of any method or process so disclosed, may be combined in any combination, except combinations where at least some of such features or steps are mutually exclusive. The protection extends to any novel one, or any novel combination, of the features disclosed in this specification (including any accompanying claims, abstract, and drawings), or to any novel one, or any novel combination, of the steps of any method or process so disclosed.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of the stated amount. As another example, the terms “generally parallel” and “substantially parallel” may refer to a value, amount, or characteristic that departs from exactly parallel by less than 14 degrees.

What is claimed is:

1. A method of implanting a cerebrospinal fluid (CSF)-access device, the method comprising:
loading the CSF-access device onto a delivery assembly, wherein loading comprises engaging an interlock of the delivery assembly, the interlock configured to hold the CSF-access device longitudinally fixed relative to the delivery assembly;
advancing the delivery assembly into the CSF space to bring a distal end of the CSF-access device into the CSF space;
releasing the interlock to allow the delivery assembly to move longitudinally relative to the CSF-device; and
withdrawing the delivery assembly from the patient leaving the distal end of the CSF-access device in the CSF space.

2. The method of claim 1, further comprising piercing a dura of the patient with the distal end of the delivery assembly.

3. The method of claim 1, wherein advancing the delivery assembly comprises advancing the delivery assembly along a guidewire.

4. The method of claim 1, wherein loading the CSF-access device onto the delivery assembly places the CSF-access device in a small-profile configuration, an outer dimension of the CSF-access device being reduced in the small-profile configuration compared to the outer dimension following withdrawing the delivery assembly from the patient.

5. The method of claim 1, wherein loading the CSF-access device onto the delivery assembly comprises pressing a distal face of the delivery assembly against a bore constriction of the CSF-access device.

6. The method of claim 1, wherein withdrawing the delivery assembly from the patient further comprises deploying, in the CSF space, a tissue anchor of the CSF-access device.

7. A method of removing an implanted CSF-access device from a patient, the method comprising:
locating a proximal hub of the implanted CSF-access device;
advancing proximally a distal end of an implant-retrieval device through a central bore of the implanted CSF-access device;
interlocking the implanted CSF-access device with the implant-retrieval device thereby fixing longitudinally the CSF-access device to the implant-retrieval device; and
withdrawing from the patient the implant-retrieval device and the implanted CSF-access device interlocked to the implant-retrieval device.

8. The method of claim 7, wherein advancing comprises piercing with a distal end of the implant-retrieval device a sealing membrane disposed on a proximal hub of the implanted CSF-access device.

9. The method of claim 7, wherein interlocking comprises engaging a first coupling feature disposed on the central bore with a second coupling feature disposed on an outer surface of the implant-retrieval device.

10. The method of claim 7, wherein interlocking comprises capturing with the implant-retrieval device a coupling feature disposed on an outer surface of the implanted CSF-access device.

11. The method of claim 7, wherein interlocking comprises engaging magnetically a first magnet disposed on the implanted CSF-access device with a second magnet disposed on the implant-retrieval device.

12. The method of claim 7, wherein interlocking comprises pressing a distal face of the implant-retrieval device against a bore constriction disposed within the central bore.

13. The method of claim 7, wherein interlocking comprises retracting a tissue anchor disposed on the implanted CSF-access device.

14. A method of sealing a CSF leakage site of a patient, the method comprising:
advancing a closure device to bring a balloon at a distal end of the closure device into the CSF space;
inflating the balloon;
releasing a tissue sealant from the closure device;
deflating the balloon; and
withdrawing the closure device from the patient.

15. The method of claim 14, wherein releasing a tissue sealant is performed both before and after deflating the balloon.

16. The method of claim 14, wherein a wall of the closure device comprises a first lateral channel fluidically connected to the balloon and a second lateral channel fluidically connected to an outflow orifice disposed on an outer surface of the closure device, and wherein releasing a tissue sealant comprises advancing the tissue sealant distally through the second lateral channel to expel the tissue sealant through the outflow orifice.

17. The method of claim 16, wherein the closure device comprises a central bore disposed radially inward of each the first and the second lateral channels.

18. The method of claim 17, wherein advancing the closure device comprises advancing the closure device along a guidewire that extends through the central bore.

19. The method of claim 17, wherein releasing a tissue sealant comprises advancing the tissue sealant distally through the central bore.
